(19) United States
(12) Patent Application Publication
(10) Pub. No.: US 2014/0256692 A1
(43) Pub. Date: Sep. 11, 2014

(54) Composition and Method for Alleviation of Pain and Inflammation

(71) Applicant: Nicholas Adam Brockman, Hawesville, KY (US)

(72) Inventor: Nicholas Adam Brockman, Hawesville, KY (US)

(21) Appl. No.: 14/097,970

(22) Filed: Dec. 5, 2013

Related U.S. Application Data
(60) Provisional application No. 61/774,321, filed on Mar. 7, 2013.

(51) Int. Cl. A61K 31/573; A61K 31/167

(52) U.S. Cl. CPC A61K 31/573; A61K 31/167
USPC 514/171

(57) ABSTRACT

A composition and method for alleviation of pain and inflammation which alleviates pain and inflammation from muscle strain, spasms, arthritis, and bursitis. The composition and method for alleviation of pain and inflammation generally includes a combination of lidocaine HCl, hydrocortisone acetate, menthol, glucosamine HCl, and a number of other ingredients. The composition described and claimed herein has been shown to alleviate pain and inflammation from injuries and conditions such as muscle strains, spasms, arthritis, and bursitis. The composition will preferably be incorporated into a topical cream which may be applied to the skin around the affected area to provide not only immediate relief from pain and inflammation, but also long-lasting relief which may last for days or even weeks.

### CROSS REFERENCE TO RELATED APPLICATIONS

I hereby claim benefit under Title 35, United States Code, Section 119(e) of United States provisional patent application Ser. No. 61/774,321 filed Mar. 7, 2013. The 61/774,321 application is currently pending. The 61/774,321 application is hereby incorporated by reference into this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to a composition and more specifically it relates to a composition and method for alleviation of pain and inflammation which alleviates pain and inflammation from muscle strain, spasms, arthritis, and bursitis.

2. Description of the Related Art

Any discussion of the related art throughout the specification should in no way be considered as an admission that such related art is widely known or forms part of common general knowledge in the field.

There is a growing need for topical creams for the treatment of pain and inflammation from injuries such as muscle strain, spasms, arthritis, and bursitis. As one gets older, it is increasingly common for joints to become inflamed, such as due to arthritis. Sports and exercise injuries are also increasingly common in a society that is growing more and more health conscious over the years. Although oral pharmaceuticals are shown to be effective for treatment of these conditions, they often carry undesirable side effects such as addiction or organ (such as liver) problems.

Because of the inherent problems with the related art, there is a need for a new and improved composition and method for alleviation of pain and inflammation which alleviates pain and inflammation from muscle strain, spasms, arthritis, and bursitis.

### BRIEF SUMMARY OF THE INVENTION

The invention generally relates to a composition which includes a combination of lidocaine HCL, hydrocortisone acetate, menthol, glucosamine hydrochloride (glucosamine HCl), and a number of other ingredients. The composition described and claimed herein has been shown to alleviate pain and inflammation from injuries and conditions such as muscle strains, spasms, arthritis, and bursitis. The composition will preferably be incorporated into a topical cream which may be applied to the skin around the affected area to provide not only immediate relief from pain and inflammation, but also long-lasting relief which may last for days or even weeks.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto. In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

### DETAILED DESCRIPTION OF THE INVENTION

### A. Overview.

The present invention is directed to a composition for the alleviation of pain and inflammation. More specifically, the composition described and claimed herein has been shown to alleviate pain and inflammation from injuries and conditions such as muscle strains, spasms, arthritis, and bursitis. The composition will preferably be incorporated into a topical cream which may be applied to the skin around the affected area to provide not only immediate relief from pain and inflammation, but also long-lasting relief which may last for days or even weeks. It should be appreciated that the present invention may be incorporated into a range of mediums, and need not necessarily be comprised of a topical cream as described herein. It should also be appreciated that the components or ingredients of the composition may vary for different applications or embodiments of the present invention.

### B. Composition.

i. Primary Ingredients.

The present invention is comprised of a composition which may be topically applied to the body to alleviate pain and inflammation. The composition's primary active ingredients include hydrocortisone acetate, lidocaine hydrochloride (lidocaine HCl), menthol, and glucosamine hydrochloride (glucosamine HCl). These four primary active ingredients, when combined in a composition such as a topical cream, will act to treat both acute or chronic pain and inflammations; particularly those associated with muscle strain, spasms, arthritis, and/or bursitis.

Hydrocortisone acetate is a steroidal drug used topically to treat skin irritation, allergic reactions, and other types of skin problems. Hydrocortisone acetate is well known as a treatment of inflammations and has been shown to treat wounds and rejuvenate human skin.

Lidocaine HCl is a drug which is generally applied topically to stop itching and pain from various skin conditions (such as eczema, burns, etc.) and to treat minor discomfort and itching caused by inflammations such as hemmorhoids. Lidocaine HCl is generally prepared in a cream, lotion, spray, foam or gel for topical application to the skin.

Menthol may also be included in the composition as a primary ingredient for its analgesic properties. In a preferred embodiment, menthol will comprise approximately 13.05% of the composition by weight.

Glucosamine hydrochloride (glucosamine HCl) is a pharmaceutical which has been shown to act as a joint degeneration retardant. Glucosamine HCl may thus be included as a secondary ingredient in the present invention to improve the composition's joint degeneration treatment properties. Glucosamine HCl will preferably comprise approximately 0.08% of the composition by weight.

The present invention will generally include between 40% and 70% by weight of hydrocortisone acetate and between 20% and 50% by weight of lidocaine HCl. In a preferred embodiment, the present invention will include approximately 45.67% by weight of hydrocortisone acetate and approximately 24.6% by weight of lidocaine HCl.

ii. Secondary Ingredients.

The composition described herein will also include one or more secondary ingredients which may be included in the composition in combination with the lidocaine HCl and the hydrocortisone acetate. By way of example and without limitation, the secondary ingredients may include such components as aloe vera, flower extract such as from heterotheca inuloides, C12-15 aklyl benzoate, coconut oil and glycerin, ceteareth-20, chondroitin sulfate, dimethicone, emu oil, glycerine, glyceryl stearate (GMS), methyl sulfonyl methane (MSM), polyethylene glycol (PEG) such as PEG-100 stearate, phenoxyethanol, ethylhexylglycerin, polysorbate 80, propylene glycol, sodium hyaluronate, and/or water as described herein.

The present invention may include aloe vera within the composition. Aloe vera is well known as a plant species which soothes inflammations such as burns, moisturizes the skin and aids in healing minor wounds. An aloe vera gel will be included as part of the secondary ingredients of the composition claimed herein to improve the soothing effects of the composition. In some embodiments, aloe vera may be omitted. In a preferred embodiment, aloe vera gel will comprise approximately 0.07% of the composition by weight.

The present invention may also include extract from flowers of the heterotheca inuloides flower (sometimes referred to as arnica inuloides). Heterotheca inuloides has been shown to have an anti-inflammatory and analgesic effect and has been used as a traditional medicine in Mexico. Extract from the flowers of the heterotheca inuloides may be included as part of the secondary ingredients of the composition claimed herein to improve the anti-inflammatory and analgesic effects of the composition. In a preferred embodiment, such extract will comprise approximately 0.38% of the composition by weight.

The present invention may also include alkyl benzoate, commonly comprised of a benzoic acid and long-chain alcohols. Alkyl benzoate has been shown to be effective both as an antimicrobial and skin conditioning agent. Preferably, C12-15 alkyl benzoate will be included as part of the secondary ingredients of the composition claimed herein to improve its antimicrobial and skin conditioning properties. In a preferred embodiment, C12-15 alkyl benzoate will comprise approximately 2.08% of the composition by weight.

The present invention may also include coconut oil, such as caprylic triglyceride oil or capric triglyceride oil. Glycerin may also be utilized. These products are commonly utilized as an emollient which has skin-repairing properties. They are commonly used in various products both as a thickener as well as to improve the moisturization and replenishment provided by the products. Coconut oils and/or glycerin may be included as part of the secondary ingredients of the composition claimed herein as a thickening agent and emollient to improve moisturization and replenishment of skin around the affected area. In a preferred embodiment, coconut oil will comprise approximately 1.43% of the composition by weight. Such a preferred embodiment may also comprise approximately 0.27% of glycerin by weight.

The present invention may also include cetostearyl alcohol or, more specifically, ceteareth-20. Ceteareth-20 is a mixture of fatty alcohols such as cetyl and staryl alcohols. Ceteareth-20 has been shown to be an emulsion stabilizer, opacifying agent, foam boosting surfactant, viscosity increasing agent, and emollient. Ceteareth-20 may be included in the present invention to improve its viscosity and texture, as well as to act as an emollient to improve moisture. Ceteareth-20 will preferably comprise approximately 2.41% of the composition by weight.

The present invention may also include chondroitin sulfate. In a preferred embodiment, porcine chondroitin sulfate will be utilized. Chondroitin sulfate has been shown to be effective as an anti-inflammatory. Chondroitin sulfate may be included in the present invention as a secondary ingredient to improve its anti-inflammatory properties. Chondroitin sulfate will preferably comprise approximately 0.15% of the composition by weight.

Dimethicone, which is a mixture of polydimethylsiloxanes and silicon dioxide, is sometimes referred to as simethicone. Dimethicone is included in the a preferred embodiment of the presently described composition to act as an antifoaming agent and carminative, as well as to provide skin protection. Dimethicone will preferably comprise approximately 0.24% of the composition by weight.

Emu oil, comprised of fat from an emu, may be included as a secondary ingredient in the composition. Emu oil has been shown to act as an emollient which heals skin. Emu oil will preferably comprise approximately 4.75% of the composition by weight.

Glyceryl stearate, sometimes referred to as glycerol monostearate or GMS, is an organic molecule used as an emulsifier. In a preferred embodiment of the present invention, glyceryl stearate SE may be included as an emulsifying agent. Glyceryl stearate will preferably comprise approximately 1.54% of the composition by weight.

Methyl sulfonyl methane, or MSM, is an organosulfur compound which is also referred to as DMSO2, methyl sulfone, or dimethyl sulfone, has been shown to have anti-inflammatory properties for joints. MSM may thus be included as a secondary ingredient in the present invention to reduce inflammation. In a preferred embodiment, MSM comprises approximately 0.03% of the composition by weight.

Polyethylene glycol, sometimes referred to as PEG, is a polyether compound which acts as a surfactant and increases lubrication. PEG may thus be included in the present invention to act as a surfactant and lubricant for the composition. In a preferred embodiment, the polyethyle glycol will be comprised of PEG-100 stearate and comprise approximately 0.24% of the composition by weight.

Phenoxyethanol is a glycol ether commonly utilized in skin creams as a bactericide and preservative. Ethyl hexylglycerin is a deodorizing agent. Phenoxyethanol and ethyl hexylglycerin may thus be included as secondary ingredients of the composition to aid in preservation and prevent formation of bacteria over time and to act as a deodorizer for the composition. Phenoxyethanol and ethyl hexylglycerin will preferably comprise approximately 1.03% of the composition by weight.

Polysorbates may also be included in the composition as an emulsifier. In a preferred embodiment, polysorbate 80, which is a nonionic surfactant and emulsifier derived from polyethoxylated sorbitan and oleic acid, will comprise approximately 0.65% of the composition by weight.

Propylene glycol is an organic compound which acts as a humectant and viscosity decreasing agent, a coupling agent, and a solvent, may be included in the composition. Propylene glycol will preferably comprise approximately 0.56% of the composition by weight.

Hyaluronan, which is sometimes referred to as sodium hyaluronate or hyaluronic acid, is an anionic nonsulfated glycosaminoglycan which has been shown to have anti-aging effects and acts as a water binding agent to fill spaces between connective fibers such as collagen and elastin in the dermis. Hyaluronan will preferably comprise approximately 0.57% of the composition by weight.

The composition may also include some quantity of water. In a preferred embodiment, water will comprise approximately 0.2% of the composition by weight.

### C. Method of Use of Preferred Embodiment

The present composition will preferably be adapted for topical application to the body. Preferably, the present composition will be comprised of a cream. The cream may be applied to portions of the body which are afflicted by pain or inflammation, such as area of skin adjacent to inflamed joints or injuries (such as burns or skin damage). The combination of primary ingredients will act to alleviate pain and inflammation in the areas adjacent to application of the cream. The secondary ingredients will improve various qualities of the cream such as its viscosity, analgesic effects, and preservation over time.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods and materials are described above. All publications, patent applications, patents, and other references mentioned herein are incorporated by reference in their entirety to the extent allowed by applicable law and regulations. The present invention may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

The invention claimed is:

1. A composition for alleviation of pain and inflammation, comprising:
hydrocortisone acetate;
lidocaine hydrochloride;
menthol; and
glucosamine hydrochloride.

2. The composition for alleviation of pain and inflammation of claim 1, wherein said composition comprises between 40% and 70% by weight of said hydrocortisone acetate.

3. The composition for alleviation of pain and inflammation of claim 2, wherein said composition comprises between 20% and 50% by weight of said lidocaine hydrochloride.

4. The composition for alleviation of pain and inflammation of claim 3, wherein said composition comprises 45.67% by weight of said hydrocortisone acetate.

5. The composition for alleviation of pain and inflammation of claim 4, wherein said composition comprises 24.6% by weight of said lidocaine hydrochloride.

6. The composition for alleviation of pain and inflammation of claim 1, further comprising one or more emulsifiers.

7. The composition for alleviation of pain and inflammation of claim 6, wherein said one or more emulsifiers are selected from a group consisting of glyceryl stearate, ceteareth-20 and polysorbate 80.

8. The composition for alleviation of pain and inflammation of claim 1, further comprising one or more emollients.

9. The composition for alleviation of pain and inflammation of claim 8, wherein said one or more emollients are selected from a group consisting of glycerin, coconut oil, ceteareth-20, and emu oil.

10. The composition for alleviation of pain and inflammation of claim 5, further comprising aloe vera, flower extract from heterotheca inuloides, C12-15 aklyl benzoate, coconut oil, glycerin, ceteareth-20, chondroitin sulfate, dimethicone, emu oil, glucosamine hydrochloride, glycerine, glyceryl stearate, menthol, methyl sulfonyl methane, PEG-100 stearate, phenoxyethanol, ethyl hexylglycerin, polysorbate 80, propylene glycol, sodium hyaluronate, and water.

11. The composition for alleviation of pain and inflammation of claim 10, wherein said composition comprises 0.07% by weight of said aloe vera.

12. The composition for alleviation of pain and inflammation of claim 11, wherein said composition comprises 0.38% by weight of said flower extract.

13. The composition for alleviation of pain and inflammation of claim 12, wherein said composition comprises 2.08% by weight of said C12-15 alkyl benzoate.

14. The composition for alleviation of pain and inflammation of claim 13, wherein said composition comprises 1.43% by weight of said coconut oil and 0.27% by weight of said glycerin.

15. The composition for alleviation of pain and inflammation of claim 14, wherein said composition comprises 2.41% by weight of said ceteareth-20 and 0.15% by weight of said chondroitin sulfate.

16. The composition for alleviation of pain and inflammation of claim 15, wherein said composition comprises 0.24% by weight of said dimethicone, 4.75% by weight of said emu oil, and 0.08% by weight of said glucosamine hydrochloride.

17. The composition for alleviation of pain and inflammation of claim 16, wherein said composition comprises 1.54% by weight of said glyceryl stearate, 13.05% by weight of said menthol, and 0.03% by weight of said methyl sulfonyl methane.

18. The composition for alleviation of pain and inflammation of claim 17, wherein said composition comprises 0.24% by weight of said PEG-100 stearate, 1.03% by weight of said phenoxyethanol and said ethyl hexylglycerin, 0.65% by weight of said polysorbate 80, 0.56% by weight of said propylene glycol, 0.57% by weight of said sodium hyaluronate, and 0.2% by weight of said water.

19. The composition for alleviation of pain and inflammation of claim 3, wherein said composition is comprised of a topical cream.

20. A method of alleviating pain and inflammation, comprising:
providing a cream comprised of hydrocortisone acetate, lidocaine hydrochloride, menthol, glucosamine hydrochloride, one or more emulsifiers, and one or more emulsion agents; and
topically applying said cream to an inflicted area of a body.
